(19) United States
(12) Patent Application Publication
(10) Pub. No.: US 2011/0287425 A1
(43) Pub. Date: Nov. 24, 2011

(54) Assays to Detect Small-Scale Mutations in Individual Cells

(75) Inventors: F. Andrew Ray, Fort Collins, CO (US); Edwin H. Goodwin, Laporte, CO (US)

(73) Assignees: KROMATID, INC., Fort Collins, CO (US); COLORADO STATE UNIVERSITY RESEARCH FOUNDATION, Fort Collins, CO (US)

(21) Appl. No.: 13/107,569

(22) Filed: May 13, 2011

Related U.S. Application Data
(60) Provisional application No. 61/334,670, filed on May 14, 2010.

(51) Int. Cl. C12Q 1/68

(52) U.S. Cl. 435/6.11

(57) ABSTRACT

Methods, compositions, and assays are described which are useful in identifying point mutations, identifying cancer cells, and diagnosing cancer.

### RELATED APPLICATIONS

This application claims priority to U.S. provisional application Ser. No. 61/334,670, entitled “An Assay to Detect Small-Scale Mutations in Individual Cells”, filed May 13, 2010, and incorporated by reference herein in its entirety.

### FIELD

This disclosure relates to cancer diagnostics, and in particular, methods, compositions, and kits for identifying point mutations and diagnosing cancer.

### BACKGROUND

The statements in this section merely provide background information related to the present disclosure and may not constitute prior art.

Early detection of cancer is a praiseworthy although difficult goal. Detecting low levels of biochemical markers, or identifying rare cancer cells, are well known problems. Less well appreciated, yet essential, is the need to minimize false positives. The most common cancer treatments—surgery, radiation, and chemotherapy—are all quite harsh on patients. Oncologists need to have a high degree of confidence in early diagnosis, especially considering tumors detected early are likely to be small and their presence difficult, or impossible to verify with other tests, before they will begin such harsh treatments based on a early detection assay.

Further effort is needed to develop more reliable diagnostic methods for early detection of cancer.

The present invention is directed toward overcoming one or more of the problems discussed above.

### BRIEF DESCRIPTION OF THE DRAWINGS

The drawings described herein are for illustration purposes only and are not intended to limit the scope of the present disclosure in any way.

FIG. 1 shows hybridization to mRNA in 8016 cells (a murine cell line) using fluorescent probes. A. is a negative control consisting of fluorescein-labeled random PNA probes. B. is a positive control fluorescein-conjugated PNA probe (green) directed against glyceraldehyde 3-phosphate dehydrogenase and a PNA-DNA probe directed against the PU.1 R235C mutation and labeled with Alexa Fluor 594-5 (red) using terminal transferase.

FIG. 2 shows competitive hybridization of fluorescent PNA-DNA probes. 8016 cells with the recurrent R235C mutation were hybridized (61° C.) with mixtures of PNA-DNA probes. The probes were both labeled using terminal transferase. The R235C PNA-DNA probe was labeled with AlexaFluor 594-5 (red) and the wild-type PNA-DNA probe was labeled with digoxigenin and detected with a fluorescein labeled antibody (green). At identical concentrations, the mutant probe signal dominates the wild-type probe signal and appears predominately red (left). As the concentration of the wild-type probe was increased (right), the green signal increased indicating the stringency used was not sufficiently high to abolish all affinity for the mismatched probe.

FIG. 3 demonstrates probe labeling. A. Oligo prior to labeling with domains color-coded as follows: The probe (black) is a sequence that is complementary to the target to be detected. Synthesis is primed by a complementary primer (dark red) base paired to an annealing site (blue) through a non-base-pairing hinge domain (orange). The template domain (green) allows incorporation of labeled nucleotides. A stop domain (red) prevents polymerization from proceeding into the probe domain (not needed if probe is PNA). B. Oligo after labeling. The light blue line represents polymerization of new DNA during which labeled nucleotides are added to the oligo. Probes with sequences complementary to the PNA sequence of the two original PNA-DNA probes may be designed as negative controls. These probes are not expected to hybridize because they have the same sequence as the PU.1 mutant and wild-type mRNAs, and are expected to demonstrate conclusively that mRNA is the hybridization target and not the gene's DNA sequence.

FIG. 4 demonstrates the use of Laser Scanning Cytometry to measure fluorescence in PNA probe competition experiments. 8016 cells were analyzed. Two PNA probes mixed in equal proportions were hybridized. One PNA probe was the exact match for the sequence around the most common point mutation (R235C), the second PNA probe was the exact match for the wild type sequence and was designed to be isothermic to the first. In these experiments, one probe was labeled with Alexa Fluor 488 and the second competing probe was not labeled. The cells were counterstained with propidium iodide in order to measure DNA content. An area containing cells was demarcated on the glass slide and analyzed using the iCys instrument from Compucyte. Green fluorescence representing probe hybridization and red fluorescence representing DNA content were acquired for each cell in the demarcated area. A. Individual cells are shown in their relative positions. Additionally each cell is color-coded based on red fluorescence and can be located using the microscope. B. The green fluorescence was integrated and mean values were recorded for all the cells or for any subpopulation. C. The DNA content—red fluorescence is a reflection of the cell cycle position of any given cell. Cells with DNA contents greater than G2+M can be eliminated by gating. D. This microscope based iCys laser scanning cytometer has 3 lasers, 3 phomultipliers, automatic focusing and motorized stage.

FIG. 5 shows PU.1_{(del/R235C)} (8016) cells hybridized to fluorophore-labeled PNA-DNA probes, either alone or with an equimolar mixture of the opposite unlabeled PNA-DNA probe. In both A. and B. the black curve represents the fluorescent probe by itself, the green curve represents inclusion of the unlabelled competitor. In A. the competitor is unlabeled wild type PNA-DNA probe and the two distributions overlap, indicating that the mutant probe remains attached to the mutant cells despite the presence of a competitor. In B. the labeled probe is WT and the competitor is unlabeled mutant PNA-DNA probe. The fluorescence is decreased significantly when the competitor is added, indicating that the unlabeled mutant probe has preferentially bound to the mutant cells. The results indicate that the mutant probe binds more efficiently to the mutant target site as expected by the higher affinity.

### SUMMARY

Provided herein are assays and associated analytical methods to detect cancer-related point mutations. The assays are amenable to high-throughput analysis to allow detection of rare cells, and permit further evaluation of a sample for multiple additional cancer-related characteristics in the same cell in order to more definitively identify the cell as having come from a tumor.

Further areas of applicability of the present teachings will become apparent from the description provided herein. It should be understood that the description and specific examples are intended for purposes of illustration only and are not intended to limit the scope of the present teachings.

As such, provided herein is a method of identifying a point mutation in a cell. The method comprises (a) providing a cell obtained from a tissue sample; (b) providing a first hybridization solution comprising probes complementary to a portion of a wild-type mRNA containing the site where the mutation is known to occur; (c) providing a second hybridization solution comprising probes complementary to a portion of mutant mRNA containing the point mutation; (d) incubating the cell with the first hybridization solution; (e) incubating the cell with the second hybridization solution; (f) detecting the amount of hybridized probe in the cell relative to a wild-type control.

Further provided are compositions comprising probes complementary to a portion of a wild-type mRNA containing the site where a mutation is known to occur and probes complementary to a portion of mutant mRNA containing the point mutation.

Still further provided are kits comprising: (a) a first hybridization component comprising probes complementary to a portion of a wild-type mRNA containing the site where the mutation is known to occur; and (b) a second hybridization component comprising probes complementary to a portion of mutant mRNA containing the point mutation.

### DETAILED DESCRIPTION

The following description is merely exemplary in nature and is not intended to limit the present disclosure, application, claims, compositions, or uses.

Provided herein are compositions, methods, and assays to detect cancer-related point mutations amenable to high throughput and able to be combined with other assays to detect cancer markers. The technology enables early and accurate diagnosis of cancers that have not manifested clinically, and promotes individualization of treatment by allowing cancer therapies to be devised for each patient based on a more precise characterization of the molecular alterations found in tumor cells.

Common point mutations that occur during neoplastic lineage evolution and in tumors can be detected using specialized PCR assays, but these assays destroy the cellular context. Cellular context includes, for example, DNA content, gene copy number, gene expression levels, cell type, and additional cancer-related mutations in the same cell in which the tested mutation occurred. Detection of point mutations on a per cell basis is necessary because cancer is the result of genome evolution within a common lineage of cells. Therefore it is important to determine if two or more oncogenic changes, any two of which are required for malignancy, are found in the same cell or in two different cells. PCR tests, for example, might identify different oncogenic mutations in a population of cells obtained from a biopsy. This might lead to a tentative cancer diagnosis, but an equally viable alternative explanation is that the mutations were present in different cells within the biopsy and cancer is not present. If cancer early detection assays are to fulfill their promise, oncologists will need to have confidence in their predictions.

The approach to improving the reliability of cancer detection assays provided herein preserves cellular context so that multiple cancer-related markers can be examined in the same cell in order to increase confidence that the cell did indeed originate from a tumor. Further provided are methods that enable identification of individual cells containing cancer-related point mutations. Still further provided are assays to detect a point mutation present only once per cell affecting a single base pair out of more than 6 billion base pairs. In a biopsy, cells containing the point mutation may occur only infrequently in an otherwise normal cell population. Thus the detection of one cell in 100,000 would represent finding one mismatched base in 10¹⁴ base pairs.

The point mutation assays can be multiplexed with detection of other cancer markers. Combined detection of oncogenic point mutations with other cell-based assays allows tumorigenic cell types to be identified at an early stage and differentiated from ‘at risk’ preneoplastic cell lineages that require one or more additional genetic alterations before they become tumorigenic.

As such, disclosed herein are methods to detect point mutations in individual cells, assays to detect point mutations in individual cells, and kits to detect point mutations in individual cells. Further disclosed are assays to detect point mutations in individual cells multiplexed with other assays, such as, for example, DNA content (ploidy), gene deletion, and immunophenotyping to identify cell type. Methods and assays disclosed herein employ flow cytometry and/or laser scanning cytometry to detect point mutations in individual cells.

### Cancer-Related Molecular Alterations

Common point mutations that occur during neoplastic lineage evolution and in tumors can be detected with great sensitivity using PCR assays. However PCR destroys the cellular context in which the mutation occurred making it impossible to determine if any particular cell having the mutation identified by the PCR test also had additional cancer-related mutations. Nor is it possible to determine that cell's DNA content, the copy number of any gene of interest, gene expression levels, cell type or other information of potential importance to establishing the cancer phenotype of that cell.

Detection of point mutations on a per cell basis is useful because cancer is the result of genome evolution within a common lineage of cells. Therefore it is useful to determine if two oncogenic changes, both required for the tumor, are found in the same cell or in two different cells. An example might be PCR tests identifying two different oncogenic mutations in a population of cells obtained from a biopsy and leading to a tentative cancer diagnosis. Another alternative is that the mutations were present in different cells within the biopsy and cancer is less likely.

It is contemplated herein that the methods and assays disclosed will be useful in identifying point mutations in a variety of genes, and particularly useful in identifying point mutations that are gain-of-function mutations such as Ras mutations.

### High-Throughput Multiplexed Single-Cell Assay

Assays provided herein are adapted to high throughput automated analysis, specifically, flow cytometry and laser scanning cytometry (LSC). The information content of the assay can be enhanced by combining detection of point mutations with a secondary marker such as DNA content (ploidy) analyses, a specific gene deletion analysis, and/or immunophenotyping with the identification of cells expressing recurrent mutations.

Her2 is most commonly amplified, mismatch repair genes are mutated, and commonly deleted genes are p53, BRCA1, BRCA2, RB, PTEN. p53 also has a recurrent gain of function point mutation as it loses tumor suppressor activity at same time it becomes oncogenic.

DNA content can be measured using a DNA dye such as, for example, propidium iodide, 7-AAD, DAPI, Hoechst 33342 trihydrochloride trihydrate, SYBR Green I, YO-PRO-1, TOTO-3, or TO-PRO-3.

Exemplary antibodies useful herein are antibodies to a protein such as Her2 or Ca125.

Laser scanning cytometry allows cells that are attached to a fixed matrix such as a glass microscope slide or multiwell plate to be detected singly, interrogated at multiple wavelengths, and analyzed automatically like a flow cytometer. Unlike cells analyzed using flow cytometry, individual cells of interest can be revisited and visually inspected.

This capability may lead to valuable insights. For example, preliminary observations suggest mRNA messages tend to accumulate in perinuclear domains. When examining heterozygotes in which one allele has a recurrent mutation and the other is wild-type, probes to the two mRNAs labeled in different colors may produce two distinct fluorescent domains.

LSC and flow cytometry both use lasers to provide intense monochromatic illumination of cells. This allows cellular constituents to be detected with higher sensitivity than in standard epifluorescence microscopy. Because each technology has substantive advantages over the other, it is probable that the detection of point mutations will evolve on both platforms. Using either technology, it is easy to imagine analyzing samples for multiple cancer-related endpoints, for example, assessing DNA content with a DNA binding dye, identifying a recurrent point mutation in one copy of a mRNA using PNA-DNA probes, and confirming expression of an altered protein (perhaps an oncoprotein) with a fluorescent monoclonal antibody. Note that PNA-DNA probes and mRNA are single-stranded and do not require thermal denaturation before hybridization. This preserves antibody detection of the oncoprotein. In LSC, the solid substrate to which cells are attached makes it easier to maintain the integrity of the cell exposed to somewhat harsh hybridization conditions, an important consideration in advancing the technology to an automated platform. LSC's ability to relocate previously analyzed cells is especially advantageous when two assays require incompatible reaction conditions.

Often such assays can be performed sequentially, and using the recorded positions, the information from each assay assigned appropriately to build a comprehensive dataset on each cell. For example, detecting a deletion of a specific locus (e.g. a tumor suppressor gene) using a FISH probe requires thermal denaturation because the target, chromosomal DNA, is double-stranded. This assay might be performed subsequent to antibody assays.

Combined detection of one or more oncogenic point mutations with other cell-based assays allows tumorigenic cell types to be identified at an early stage and differentiated from ‘at risk’ preneoplastic cell lineages that require one or more additional genetic alterations before they become tumorigenic. These assays are sensitive enough for early detection of rare cells containing cancer-associated mutations, and importantly improves the cancer diagnostic capability of the assays by additionally demonstrating that these rare cells either do or do not have other characteristics associated with a cancer cell. This last point bears some emphasis—in traditional assays, early diagnosis is not necessarily accurate diagnosis. In many cases it is unlikely that an early diagnosis of cancer based on a molecular assay could be confirmed by more traditional, presumably more reliable, assays such as x-rays, MRI etc. because the tumor would be too small. Oncologists must decide whether to begin cancer therapy based solely on the result of a molecular assay. If the detected mutation occurred in preneoplastic cells rather than a true tumor, initiation of cancer therapy could cause unnecessary physical and psychological harm to the patient. It might also fail to eradicate the preneoplastic cell lineage, and considering the DNA-damaging effects of some anticancer drugs, might instead generate new mutations in these preneoplastic cells driving them closer to malignancy. On the other hand, choosing not to begin treatment greatly limits any possible benefit from early diagnostic assays. As such, early diagnosis on the basis of a molecular assay carries an exceptionally high requirement to minimize both false positives and false negatives.

The approach provided herein tests for multiple cancer-related molecular characteristics in each analyzed cell, and requires that before any cell is labeled as a potential cancer cell it must test positive for all of the cancer-related characteristics. The probability that a potential cancer cell identified by the assay is a true cancer cell greatly increases with each cancer-related characteristic added to the assay. At the same time, any cell failing to contain as few as one of the tested cancer-related characteristics can definitively be labeled as not being a cancer cell.

Another advantage of the methods and assays provided herein is the aspect of individualization of treatment by devising a cancer therapy for each patient based on a more precise characterization of the molecular alterations found in the tumor cells. For example, the type of treatment a subject receives can depend on the type of ras mutation, e.g. H-ras, K-ras, or P-ras.

In other embodiments, identification of preneoplastic lesions can implicate preemptive treatments targeting therapies to precancerous cells.

### FISH

Fluorescence in situ hybridization (FISH) is based on the principle that allows stable binding between the two strands of the DNA double helix, i.e. hydrogen bonds formed between complementary nucleic acid bases. In general, two nucleic acid polymers having complementary bases will pair (bind or hybridize) at temperatures below a critical “melting” point. In practice, FISH uses a short polymer, typically DNA, to which one or more fluorescent molecules have been attached to create a “probe” that hybridizes to, and identifies the location of, a “target” sequence. Typically the target is a DNA sequence within a metaphase chromosome. FISH has a large, and expanding, number of applications in biomedical research and medicine.

### Probes

Peptide nucleic acid (PNA) probes are synthetic DNA analogs in which the phosphodiester backbone is replaced by repetitive units of N-(2-aminoethyl) glycine to which the purine and pyrimidine bases are attached via a methyl carbonyl linker.

PNA probes have superior hybridization characteristics, including the ability to distinguish a single-base mismatch from a wild-type nucleic acid. However, labeling procedures for PNA probes are more difficult, costly, and less effective than procedures to label similar DNA probes. Described herein are PNA-DNA chimeric oligonucleotide probes that retain PNA's mismatch discrimination but also allow labeling with reagents commonly used for DNA probes. PNA-DNA probes are exceptionally bright because multiple fluorescent molecules can be attached to each probe molecule, and a wide choice of fluorophores is available for labeling.

In contrast, commercially available PNA probes have few labeling choices and typically no more than one fluorescent molecule is attached to each probe molecule. Even with these bright probes it is unlikely that a single-base mismatch could be detected consistently on a per cell basis by hybridizing to genomic DNA. However, transcription of the mutation into mRNA, a step necessary for the expression of the cancer phenotype, increases the number of target molecules per cell by hundreds.

Probes are designed such that the melting temperature between the probes to the wild-type sequence and the probes to the mutant sequence differ by no more than about 1° C.

Automation of the process permits discovery of rare cancer cells in a sample of mostly normal cells. Two high-throughput platforms, flow cytometry and laser scanning cytometry, are suitable in detecting point mutations, for example, with PNA-DNA probes.

As such, assays provided herein surprisingly enable earlier and more reliable detection of tumors, as well as the capacity to better define tumor heterogeneity and molecular characteristics. This is a substantial improvement in the diagnosis and treatment of cancer.

It is contemplated herein that a variety of probes are useful in the methods and assays described. Illustrative probes include, but are not limited to, PNA probes, LNA probes, modified RNA probes, modified DNA probes, chimeric PNA-DNA probes, chimeric modified RNA-DNA probes, chimeric LNA-DNA probes, chimeric modified DNA-DNA probes, or mixtures thereof.

Making quality probes is an essential part of FISH.

### Locked Nucleic Acid™ (LNA®) Probes

LNA is a nucleic acid analog that contains a 2′-O, 4′-C methylene bridge. This bridge restricts the flexibility of the ribofuranose ring and locks the structure into a rigid C3-endo conformation, conferring enhanced hybridization performance and exceptional biological stability. LNA fluorescent probes can be used for quantification, melting curves profiles, as well as in singleplex, multiplex, or high-throughput screening assays.

### Peptide Nucleic Acid (PNA) Probes

Synthetic polymers (called oligomers) composed of either altered bases or backbone linkages not found in nature have been created that have hybridization properties superior to DNA. Most notable is peptide nucleic acid in which the sugar-phosphate background has been replaced with a peptide linkage. In natural DNA, the charged phosphate groups on opposite strands repel one another partially destabilizing the double helix (1). The peptide linkage of PNA is uncharged so PNA probes bind more tightly. As a result, a PNA probe is shorter than a DNA probe having the same melting temperature, so a single base mismatch more strongly destabilizes PNA probe hybridization. This property can be used to discriminate between target sequences that differ by as little as a single nucleotide (2).

### Limitations of Probes for FISH

PNA, LNA, etc. can be labeled with one or possibly two fluorophores during synthesis. For some applications this would be sufficient. The chimeric probes can be more heavily labeled, but heavy labeling is not a necessary condition for the procedure to work. The ability to heavily label probes is useful when there are few copies of the target sequence.

PNA probes are small, expensive oligomers costing more than 100 times as much as conventional DNA probes. Thus they may be cost-prohibitive in some embodiments. Although PNA probes produce bright signals with little non-specific background binding, detection of a single labeled PNA probe molecule is not possible, but detection of gene-sized target sequences covering 2,000 base pairs has been accomplished (3). Thus PNA probes are useful for detecting targets consisting of repetitive sequences clustered at centromeres and telomeres, but they are not practical for detecting low copy number targets. Part of the problem is that PNA probes available from commercial venders attach only one or two fluorophore molecules per probe molecule, and there are only a limited number of fluorophores available for labeling.

### Increasing Probe-Labeling Flexibility

Chimeric probes provide a means of increasing the number of fluorescent tags per molecule. For example, PNA-DNA oligos are synthesized such that an OH group on the DNA molecule serves as a substrate for extension by terminal transferase, thus permitting the DNA molecule to be fluorescently tagged.

The following example gives an indication of how this strategy can overcome PNA's limitations. A typical commercially synthesized PNA probe is labeled with a single fluorescent molecule like fluorescein.

Using PNA-DNA, 10 or more fluorescent nucleotides can be added to each probe molecule. Moreover a bright fluorophore like Alexa Fluor 594-5, ˜5× fluorescein's brightness, can be used in place of fluorescein. In this example, the probe is potentially 50 fold brighter than the commercial PNA probe, and much less probe will be required to obtain similar FISH signals. The cost per use scales inversely to the increase in brightness, and the technology permits labeling with any fluorophore available for DNA probes. Thus both limitations of PNA probes (expense and limited labeling options) are overcome.

Probes can be labeled with any one of a variety of detection tools, for example, a fluorescent tag, a biotinylated tag, or a hapten.

Overcoming Low Gene Copy Number Limitation: Probing mRNA in Intact Cells

Oncogene transcription is necessary to establish a malignant state, and also has the effect of amplifying the number of hybridization targets. A method to overcome low gene copy number is to select a probe's sequence so that it pairs with the gene's messenger mRNA. A published study, in which Gamma-globin mRNA was detected with fluorescent PNA probes using fluorescence microscopy and flow cytometry (4), demonstrates the method is feasible as long as mRNA is protected from degradation. With multiply labeled PNA-DNA chimeric probes, even poorly expressed low copy number genes are expected to be detectable.

Designing PNA-DNA Probes to Detect Mismatches in mRNAs

Illustratively, if the PNA portion of a PNA-DNA probe is designed to discriminate single base pair differences and the 3′ DNA end is used to increase labeling flexibility, then bright probes that allow point mutations to be detected become possible. The probes detect single-base mismatches in expressed mRNAs, thus overcoming the copy number limitation. Sample analysis time is greatly reduced (and throughput increased) with laser scanning or flow cytometry.

Methods of Identifying a Point Mutation, Detecting a Cancer Cell, and/or Diagnosing Cancer

It will be apparent to one of skill in the art that the steps provided herein can be performed in a variety of sequences, and in some instances one or more steps can be combined into one step.

In one embodiment, the steps comprise the following: Hybridize a mixture of labeled and unlabeled probes in approximately equal proportions. Record fluorescence intensity from individual cells and their positions. Strip the first set of probes. Hybridize a second set of probes with the label reversed. Record fluorescence intensity from the same cells. From the relative intensities, and in comparison to positive and negative controls, it is possible to determine if any cell in the test sample has the mutation. This embodiment can be combined with fluorescence microscopy for detection.

In another embodiment, the steps comprise the following: Label both wild-type and mutant probes in different colors (e.g., red and green). Combine the probes in approximately equal proportions and perform a single hybridization. Measure fluorescence intensities from individual cells, and compute the fluorescence intensity ratios for each cell. In comparison to positive and negative controls, it will be possible determine if any cell in the test sample has the mutation. This method is compatible with flow cytometry and laser scanning cytometry.

In still another embodiment, the steps comprise the following: Label wild-type probes and hybridize unlabeled probes to cell sample to be tested. Measure fluorescence intensity and from relative intensities and comparison to positive and negative controls, determine if there is a decrease in intensity which is indicative of a cell containing the mutation.

Illustratively, the method comprises (a) providing a cell obtained from a tissue sample; (b) providing a first hybridization solution comprising probes homologous to a wild-type mRNA from a gene known to be susceptible to a recurrent single point mutation; (c) providing a second hybridization solution comprising probes homologous to mRNA from the gene containing a known recurrent single point mutation; wherein the probes from at least one of steps (b) and (c) are labeled; (d) incubating the cell with the first hybridization solution; (e) incubating the cell with the second hybridization solution; (f) detecting the amount of hybridized labeled probe in the cell relative to a wild-type control; wherein a change in amount of hybridized labeled probe relative to a wild-type control identifies the presence of a point mutation in the gene.

In some aspects, the steps of hybridization of the mutant probes and hybridization of the wild-type probes are performed sequentially. In this case, the step of detecting the signal can be performed after each hybridization step or once after both hybridization steps have been performed. In other aspects, the hybridization of the wild-type probes and mutant probes are performed simultaneously. In the latter case, both probes are combined into the same hybridization solution.

In some embodiments, the method further comprises providing a secondary marker for a cancerous cell, contacting the cells with the marker, and detecting the secondary marker. The secondary marker can be any marker useful in making a diagnosis or useful in identifying a cancer cell. Exemplary secondary markers include a DNA dye, a labeled antibody to a protein of interest, or a DNA probe.

Comparative controls can be internal controls, e.g. cells from a biopsy that are known to be wild-type cells, or external controls, e.g. a slide with wild-type cells and a slide with known mutant cells used to compare to a similarly treated slide containing sample cells with an unknown cell population.

### Compositions

Provided herein are compositions useful in the methods and assays described. An illustrative composition comprises labeled probes complementary to a portion of a wild-type mRNA containing the site where a mutation is known to occur and unlabeled probes complementary to a portion of mutant mRNA containing the point mutation. In some aspects, the probes are chimeric PNA-DNA probes. The PNA portion of the probe can be between 10 and 20 nucleotides. The DNA portion of the labeled probe is a substrate for extension by an enzyme for labeling. The probe can be labeled with a fluorophore or any other tag useful in flow cytometry or laser scanning microscopy.

### Kits and Assays

Compositions, methods, and assays provided herein are useful as kits. As such, provided herein are kits for detection of a cancer cell, for diagnosing cancer, or for identification of a point mutation. An illustrative kit comprises (a) a first hybridization component comprising probes complementary to a portion of a wild-type mRNA containing the site where the mutation is known to occur; and (b) a second hybridization component comprising probes complementary to a portion of mutant mRNA containing the point mutation. The kit can further comprise (c) a third component comprising a secondary marker for a cancerous cell.

While the invention has been particularly shown and described with reference to a number of embodiments, it would be understood by those skilled in the art that changes in the form and details may be made to the various embodiments disclosed herein without departing from the spirit and scope of the invention and that the various embodiments disclosed herein are not intended to act as limitations on the scope of the claims.

### EXAMPLES

The following examples are provided for illustrative purposes only and are not limiting to this disclosure in any way.

### Example 1

### Selection of Sequences

Development and validation of a cellular mutation assay uses competition experiments between probes differing by single nucleotides. Using a cell line with a homozygous point mutation allows unambiguous interpretation of such experiments. In certain strains of mice, ionizing radiation is a strong inducer of acute myelogenous leukemia. Approximately 90% of the tumors isolated from these mice are found to have a large deletion in one copy of chromosome 2 (5). The deletion encompasses the PU.1 gene. Strikingly the second allele of PU.1 is found to contain a point mutation in the DNA binding domain of the remaining PU.1 gene in 80% of the mice with the PU.1 deletion. The most common point mutation is R235C and results from a single base change.

An AML cell line (8016) with the deletion and the most common point mutation, provided by Dr. Simon Bouffler, was used to develop the mutation assay because it is homozygous/null for the PU.1 gene, PU.1(del/R235C), and the copy number of the mutated gene is one. The cell line is routinely cultured in RPMI 1640 media (Hyclone) supplemented with 10% fetal bovine serum (FBS), 1% penicillin and 1% streptomycin. These cells were grown in an incubator at 37 degrees Celsius and passaged an average of 2.5 days or upon confluence, whichever comes first.

### Preparation of Probes

Design of Probes for Detecting Point Mutations in mRNA.

Chimeric probes composed of PNA for excellent mismatch discrimination and DNA for superior labeling were designed to detect a common point mutation in mouse AML and to the human ras oncogene.

General Probe Design for mRNA Detection.

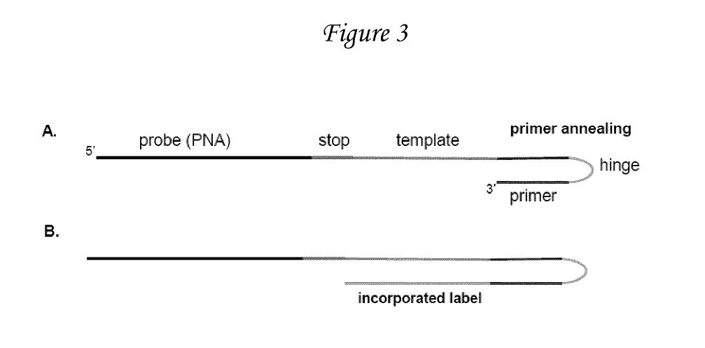

Probes were designed in pairs, with one probe homologous with a recurrent point mutation, and the second probe homologous to the wild-type sequence. The PNA portion of the probes was designed to be complementary to the gene's mRNA, and hybridized to a sequence that spans the recurrent point mutation or corresponding wild-type base so as to place this base in the central region of the probe. The PNA portion was 16-17 nucleotides unless the sequence was unusually AT rich. It is anticipated, however, that the PNA portion of the probe can be as short as 10 nucleotides and as long as 20 nucleotides, and any nucleotide length therein. The paired probes had closely matched melting temperatures, which is an important consideration for point mutation detection. The DNA portion of the chimeric probe provided a 3′ OH terminus that was a substrate for extension by a variety of enzymes for the purpose of labeling. The proprietary hairpin design shown in FIG. 3 was used to enable a controlled number of fluorophores to be added to the probe. This design produced bright probes labeled with multiple fluorescent molecules without sacrificing the ability to precisely quantify fluorescence as happens when terminal transferase adds a variable number of labeled nucleotides.

### Primer Annealing

3′ primer

5′ probe (PNA) stop template

Hinge

### Example 2

### Hybridization

Optimizing Mismatch Discrimination Using PNA-DNA Probes Hybridized to Cells with Point Mutations:

The hybridization conditions that allow a precisely matched probe to best outcompete a probe with a one base mismatch for binding to a chromosomal target sequence were determined. Cells were taken directly from flasks and transferred to centrifuge tubes. The concentration of the sample was analyzed using the Countless Cell Counter (Invitrogen). Once the concentration was about 1×10̂5 cells per ml, they were applied to Superfrost micro slides (VWR) and incubated in RNase Zap (Ambion) for 15 minutes. Slides were then sterile rinsed, and once dried were cytospun (cytocentrifuged). After experimentation, a rate of 800 RPM for a time of 4 minutes were found to be the best conditions for the 8016 cell line. Cells were pre-fixed using 50 μl of 4% paraformaldehyde, allowed to dry for at least one hour, then were fixed using 4% paraformaldehyde for 10 minutes followed by immersion in 1×PBS for 5 minutes. Immediately afterward the slides were immersed in 75%, 85%, and 100% EtOH for 2 minutes each, respectively, to dehydrate them. After air drying, the slides were stored at 4 degrees Celsius until they were ready to be used for hybridization.

PU.1(del/R235C) cells were obtained from Dr. Bouffler, and before being used, the cells were characterized to confirm the presence of the R235C point mutation by PCR amplification of a portion of the gene around the mutation by using a high fidelity thermostabile polymerase and sending the product to a DNA sequencing service. Deletion of the other PU.1 allele was confirmed by FISH analysis using a BAC probe as described by Peng et al. (6).

PU.1 expression varies in different myeloid lineages during differentiation. Preliminary results using the wild-type PU.1 probe hybridized to normal mouse bone marrow cells indicate a wide range of expression of PU.1 from no detectable expression to very high expression. Fibroblasts were used as a negative control because they do not express PU.1. Mutant and wild-type probes can by design have closely matched melting temperatures for their respective targets, however conditions for optimal mismatch detection need to be determined empirically. The conditions to be optimized are probe concentration, hybridization time, and hybridization stringency. Formamide concentrations were fixed at 70% as is required for PNA probes, and hybridization stringency was adjusted by varying temperature. Conditions for the wash steps were kept constant. The goal of these experiments was to determine conditions that allow the best discrimination between two cell samples, one in which the probe precisely matched the target sequence and the other in which the target had a one base mismatch. An alternate strategy, in which competing probes were hybridized at low stringency followed by removal of the mismatched probe through adjustment of wash stringency, was also tried. Wild-type and mutant probes were labeled with the same fluorophore and labeling was performed under identical reaction conditions. Hybridizations used equimolar concentrations of fluorophore-labeled mutant probe mixed with unlabeled wild-type probe or vice versa. Initially, a single probe mixture concentration and hybridization time was chosen. When stringency had been optimized, this parameter was fixed and then limited evaluation of varying probe concentration and hybridization time (half and twice the initial values) was performed. In order to make “optimization” a quantitative exercise, MetaSystems software was used to measure fluorescence intensity from 25 cells on each of three microscope slides for cells both with and without the R235C mutation. Sample means and standard deviations were calculated, and from these numbers the t statistic and p value were computed. Optimizing experimental conditions (hybridization or wash stringency) is then equivalent to minimizing p. The value of this procedure is that it allows us to define “optimal” in a meaningful way by equating it with the highest level of a statistically significant difference between the signal intensities measured in two samples, one in which all cells contain the mutation (PU.1(del/R235C) cells) and the other representing background in which no, or very few, cells contain the mutation (normal fibroblasts).

Hybridization temperature was systematically varied until an optimal point was reached based on a statistical evaluation where a precisely matched probe best competed with a mismatched probe. Fluorescence intensities were recorded from 25 cells on each of 3 microscope slides for each cell pair (with and without the R235C mutation), and a minimum of 5 temperatures spanning the calculated melting temperature were examined. Then half and double the probe concentration and hybridization time were evaluated for further optimization.

Hybridization conditions described herein can be varied in stringencies, times, and temperatures, for example, by as much as +/−5% of the listed value. Thus, as used herein, the term “about” indicates the following value is inclusive of values within the range of +/−5% of the listed value.

### Example 3

### Detection

Detection of a Mutation in a PU.1(del/R235C) Cell Line Using a PNA-DNA Probe.

Wild-type and mutant PNA-DNA probes were designed and synthesized. Biosynthesis Inc. synthesized the probes, which were sent in a lyophilized form. The PNA portion of the probes was complementary to a sequence around the R235C mutation. Wild-type and mutant probes differed by a single base and were designed to have the same melting temperature for their respective target sequences. The probes were solubilized in dH2O. For application purposes, the PNA probes were diluted into a 100 μM stock solution. The working probes were diluted into 10 μM aliquots from the stock aliquots, and both aliquots were stored in 20 degrees Celsius until ready for use. PNA probes were labeled using an end labeling reaction with terminal transferases. The dUTPs covalently linked to fluorophores were labeled at the 3′ end of our probe. With the probes, there was a 5× terminal transferase buffer, CoCl2 (25 mM), dUTP (1 mM) conjugated with fluorophores Alexa 488 or Alexa 594 (Invitrogen), dATP (10 mM), ddH2O and finally terminal transferase enzyme. The mixture was incubated for at least two hours at 37 degrees Centigrade in a thermocycler. Agarose gel electrophoresis with an EtBr containing agarose gel was used to visualize incorporation of the fluorophores onto the probes. A smear present above the unlabeled PNA band indicated efficient labeling.

The probes were directly hybridized to the slides in the following manner. The hybridization mix contained a 50% formamide mixture and the labeled PNA probes. 14 μl of the hybridization mix was added to each slide, and the cells were hybridized for at least two hours at 55 degrees Celsius.

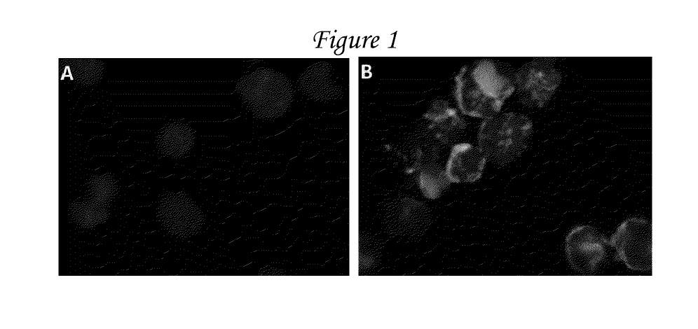

A kit produced by DAKO (K5201) intended for use with diagnostic PNA probes labeled with fluorescein was obtained. The kit's intended use is to detect RNA sequences on cell smears or tissue sections. It contains appropriate PNA controls to be used to compare to diagnostic PNA probes. The positive control PNA probe with an attached fluorescein is targeted to GA3PDH mRNA. The negative control is a mixture of fluorescein-labeled random PNA probes. The R235C PNA-DNA probe was labeled with Alexa Fluor 594-4 using terminal transferase. The cell nuclei were counterstained with DAPI. Fluorophore incorporation was monitored by agarose gel electrophoresis followed by image analysis of the gels. 8016 PU.1(del/R235C) cells were attached to microscope slides using a cytocentrifuge, fixed using 4% paraformaldehyde and hybridized at 61° C. Cells were hybridized using either the negative control PNA or a 1:1 mixture of the GA3PDH positive control PNA and the Alexa Fluor 594-5 labeled R235C PNA-DNA probe. Results showing detection of GA3PDH (green signal) and PU.1 (red signal) are shown in FIG. 1B. Virtually no green fluorescence was detected in negative control cells (FIG. 1A). RNase pretreatment of cells abolished fluorescent signals as expected (not shown).

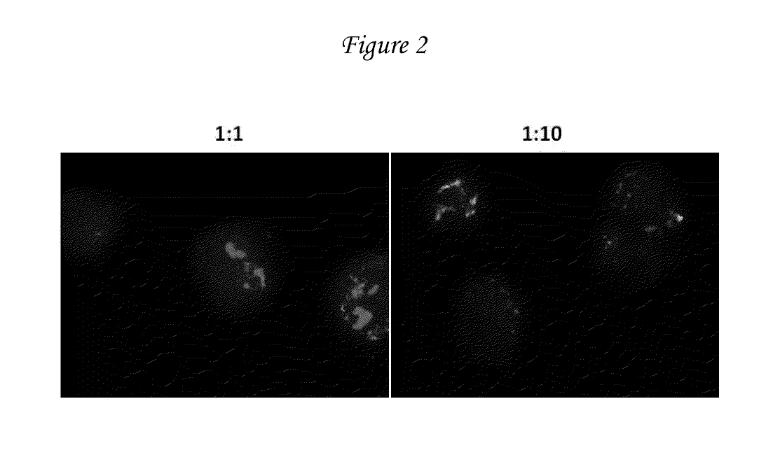

In another set of experiments, the mutant probe labeled with Alexa Fluor 594-5 (red fluorescence) was mixed with the corresponding wild-type probe labeled with Alexa Fluor 488 (green fluorescence derived from fluorescein-labeled anti-digoxigenin antibody). Both probes were designed to have the same melting temperatures to their perfectly matched targets. PNA-DNA probe mixtures were hybridized to 8016 PU.1(del/R235C) cells. When either the red (mutant) or the green (wild-type) probes were reacted singly with cells 8016 PU.1(del/R235C) cells, they produced either bright red or green perinuclear signals, respectively. The wild-type probe with a single-base mismatch was expected to hybridize to the cells with the mutation at the hybridization temperature that was used. When the two probes were mixed in equal parts, i.e. 1:1, they produced a bright red signal. This result is consistent with the conclusion that the affinity (perfectly matched) mutant probe effectively competed against the wild-type probe with the lower affinity (one base mismatch). It is also possible that the brighter red fluorescence observed in FIG. 2 is due to the brighter fluorophore 594-5. The results suggest that the probe exactly matching the mRNA expressed in the cells has a competitive advantage.

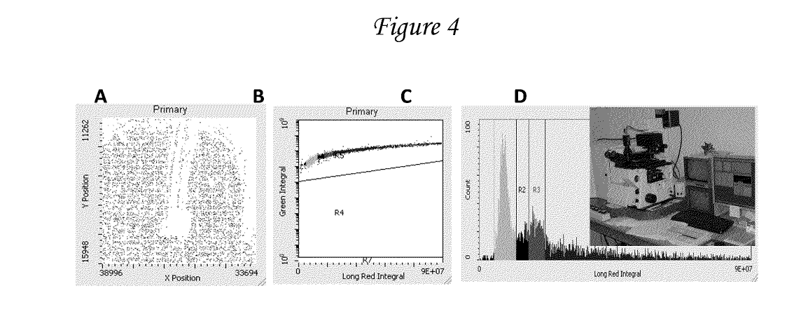

FIG. 4. Laser Scanning Cytometry was used to measure fluorescence in PNA probe competition experiments. 8016 cells were analyzed. Two PNA probes mixed in equal proportions were hybridized. One PNA probe was the exact match for the sequence around the most common point mutation (R235C), the second PNA probe was the exact match for the wild type sequence and was designed to be isothermic to the first. In these experiments, one probe was labeled with Alexa Fluor 488 and the second competing probe was not labeled. The cells were counterstained with propidium iodide in order to measure DNA content. An area containing cells was demarcated on the glass slide and analyzed using the iCys instrument from Compucyte. Green fluorescence representing probe hybridization and red fluorescence representing DNA content were acquired for each cell in the demarcated area. A. Individual cells are shown in their relative positions. Additionally each cell is color-coded based on red fluorescence and can be located using the microscope. B. The green fluorescence was integrated and mean values were recorded for all the cells or for any subpopulation. C. The DNA content—red fluorescence is a reflection of the cell cycle position of any given cell. Cells with DNA contents greater than G2+M can be eliminated by gating. D. This microscope based iCys laser scanning cytometer has 3 lasers, 3 phomultipliers, automatic focusing and motorized stage.

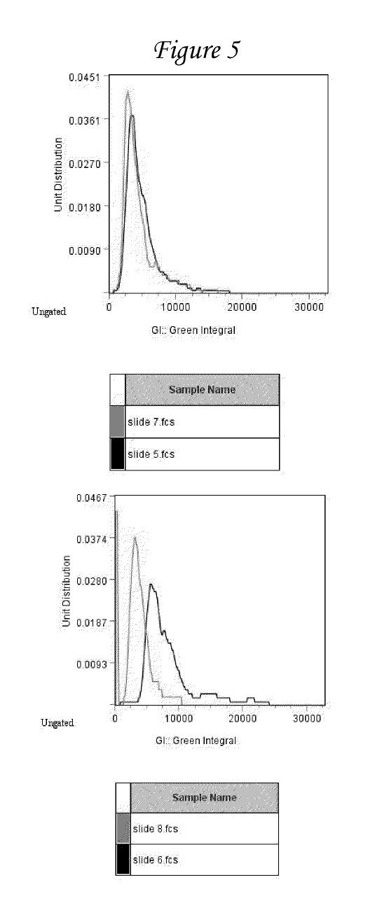

FIG. 5. PU.1_{(del/R235C)} (8016) cells were hybridized to fluorophore-labeled PNA-DNA probes, either alone or with an equimolar mixture of the opposite unlabeled PNA-DNA probe. In both A. and B. the black curve represents the fluorescent probe by itself, the green curve represents inclusion of the unlabelled competitor. In A. the competitor is unlabeled wild type PNA-DNA probe and the two distributions overlap, indicating that the mutant probe remains attached to the mutant cells despite the presence of a competitor. In B. the labeled probe is WT and the competitor is unlabeled mutant PNA-DNA probe The fluorescence is decreased significantly when the competitor is added, indicating that the unlabeled mutant probe has preferentially bound to the mutant cells. The results indicate that the mutant probe binds more efficiently to the mutant target site as expected by the higher affinity.

| | Melting |
|---|---|
| | Temperature |
| Probe Sequence | (degrees C.) |
| PU.1 mouse WT PNA-DNA- PNA portion: GCC | 60° |
| GTA GTT GCG CAG C-start DNA portion GAT | |
| TAT TAT TAT TAC CAC TCA GAG GTT TTT | |
| TCC TCT GAG TGG (NOTE THIS DNA | |
| SEQUENCE WILL BE COMMON TO ALL | |
| PROBES and will be henceforth denoted as —DNA) | |
| PU.1 R235C mouse (most common) GCT GCG CAA | 60° |
| CTA CGG C-DNA | |
| PU.1 R235H mouse (second most common-do not have | 60° |
| cell line) TTG CCG TAG TTG TGC AGC-DNA | |
| PU.1 WT sense strand mouse- negative control will not | 60° |
| bind to message GCT GCG CAA CTA CGG C-DNA | |
| KiRas human TGC CTA CGC CAC CAG C-DNA | 60° |
| KiRas human TTG CCT ACG TCA CCA GCT-DNA | 60° |

TABLE 1
Summary of three experiments using laser scanning cytometry.
PU.1 R235C mutant cells (8016) Mouse bone marrow cells-WT

| | | | | | | |
|---|---|---|---|---|---|---|
| PNA- | Exp. 1 | Exp. 2 | Exp. 3 | Exp. 1 | Exp. 2 | Exp. 3 |
| DNA | Ave. | Ave. | Ave. | Ave. | Ave. | Ave. |
| Probes | FL(%)1 | FL(%)1 | FL(%)1 | FL(%)1 | FL(%)1 | FL(%)1 |

| | | | | |
|---|---|---|---|---|
| R235C* | 24.0 | 8.9 | 2.4 | 2.3 |
| R235C*/ | 22.7(5.0) | 8.3(6.7) | 0.9(62.5) | 1.6(30.4) |
| WT 1:1 | | | | |
| R235C*/ | | 0.4(83.3) | 0.4(82.6) | |
| WT 1:10 | | | | |
| WT* | 46.9 | 17.5 | 2.4 | 5.3 |
| WT*/ | 20.4(56.5) | 11.7(33.1) | 1.3(45.8) | 4.2(20.8) |
| R235C 1:1 | | | | |
| WT*/ | | 0.2(91.7) | 1.5(71.7) | |
| R235C | | | | |
| 1:10 | | | | |

*Indicates end labeling with AlexaFluor 488. 1- Average green fluorescence (percentage decrease compared to labeled probe alone).

### Quantitative Fluorescence Intensity Threshold Determination Used to Maximize Discrimination Between Mutant and Wild-Type Expressed Alleles.

A quantitative procedure to set fluorescence intensity thresholds that determines whether or not a cell should be scored as having an expressed mutant allele, an expressed wild-type allele, or no expression of the gene is contemplated.

Using the optimal hybridization conditions identified above, the two probe mixtures were hybridized to 8016 PU.1(del/R235C) AML cells (positive control for R235C mutation), normal mouse bone marrow cells (positive wild-type control), and normal mouse fibroblasts (mutant and wild-type negative control). Fluorescence intensities from 25 cells on each of three slides were recorded from AML and normal bone marrow cells for each probe mixture. Although all cells are positive for the mutation, some cells that appear negative have been observed using fluorescence microscopy. LSC technology allows the cell-cycle position to be determined based on DNA content analysis. It is hypothesized that cells that are not fluorescent in this population will be mitotic cells that fall into the G2/M compartment. Because LSC allows visualization of cells of interest, the discrimination between G2 and M cells can be determined.

A quantitative procedure to set fluorescence intensity thresholds corresponding to whether or not a cell is scored as having an expressed mutant allele, an expressed wild-type allele, or no expression of the gene was established. The mathematical procedure was based on the chi-squared test for a statistically significant difference in proportions. Data for the labeled R235C probe/unlabeled wild-type probe mixture was used to assign cells to one of two groups, mutant cells or non-mutant cells (wild-type expression and no expression). At first, a fluorescence intensity threshold was chosen somewhat arbitrarily in order to decide into which group a cell should be placed. This was done for both the mutant cell and the normal bone marrow cell data. The relative proportions of mutant and wild-type cells were calculated for the two datasets, the chi-squared test applied, and value for p found. Then using a software program written by the inventors, the fluorescence intensity threshold was systematically varied, proportions calculated, and p recomputed until the minimum value of p was found. This procedure settles on a threshold that maximizes the level of significance in the difference in proportions given the data that was actually recorded. If the threshold had been set any lower, some normal cells with fluorescence intensities on the upper end of the distribution might be scored as mutants. Conversely, had the threshold been set higher, some less fluorescent mutant cells might be scored as normal.

The procedure above was repeated with data for the unlabeled R235C/labeled wildtype probe mixture to establish a fluorescence intensity threshold for scoring a cell as wild-type. Any fibroblasts identified as fluorescent with either probe mixture were visually examined and served as the baseline for scoring false positive cells.

The next set of competition experiments involved labeling wild-type and mutant probes with different fluorophores having well separated fluorescence emission spectra. Equimolar mixtures of the two probes were hybridized at the optimal stringency determined above to 8016 PU.1 (del/R235C) AML cells, normal bone marrow cells, and fibroblasts. Data was collected at both emission wavelengths. The procedure described above was used to set a fluorescence intensity threshold corresponding to what should be scored as a mutant cell, and then used again to set a normal cell threshold, each time using data for the appropriate probe's label. These experiments may provide the conditions useful for later experiments such as determination of the frequency of detection in a mixed population and later experiments using cells that are heterozygous.

Fluorescence intensities were recorded from 25 cells on each of 3 microscope slides from mutant, normal myeloid and fibroblast cells for 3 probe mixtures (labeled mutant/unlabeled wild-type, unlabeled mutant/labeled wild-type, and both labeled with different fluorophores). A quantitative procedure based on the significance of a difference in proportions was used that chooses the best fluorescence intensity thresholds needed to decide if a cell should be scored as having an expressed mutant allele, an expressed wild-type allele, or no expression of the gene.

### Automation and Quantification Using Cytometry (LSC)

Mutant and normal cells were hybridized in solution to a mixture of two labeled probes. The point mutation detection assay was adapted to automated Laser Scanning Cytometry. Next, the assay was adapted to flow cytometry and compared to LSC. Analysis time for a chosen number of cells, ability to discriminate between mutant and normal cells, and the fraction of analyzed cells that are non-informative were compared to LSC. Cells identified as mutant or normal were flow sorted, then DNA sequencing was used to confirm that the probes are detecting the correct sequences.

8016 PU.1(del/R235C) cells, mouse bone marrow cells, and fibroblasts were adhered to glass slides and hybridized with mutant and wild-type PNA-DNA probes using the stringent conditions developed using fluorescence microscopy. The slides were analyzed by Dr. Lehman at the Brody School of Medicine at Eastern Carolina University. Dr. Lehman is an expert in both flow cytometry and laser scanning cytometry and provided invaluable advice. Dr. Lehman's group at Eastern Carolina University has a Compucyte i-Cys LSC instrument.

Experiments were conducted using the CSU Flow Cytometry Analysis and Cell Sorting Facility on a MoFlo cytometer (Dako Colorado, Inc.) that had the capacity to perform 9-color (3 laser) analysis and sort up to 4 subpopulations simultaneously. The strategy for adapting the technique to a flow-based assay is to start with the fixation and hybridization conditions that were used successfully by Larsen et al. for flow detection of mRNAs using fluorescent PNA probes (4). A mixture of mutant and wild-type probes labeled with different fluorophores were used in competitive hybridization experiments as described above using LSC. The results were then compared between the two platforms. The endpoints for comparison are 1) analysis time for a chosen number of cells, 2) ability to discriminate between mutant and normal cells as determined from the best p values computed in the chi-squared test for proportions, and 3) the fraction of analyzed cells that are non-informative, i.e. testing as both mutant and normal. A major advantage of flow cytometry is the ability to sort subpopulations. The mixing experiment outlined above, in which 8016 PU.1(del/R235C) cells were mixed with normal bone marrow cells and then hybridized to the two probes as a test of rapid screening for cells containing point mutations, was repeated using flow cytometry. In this version of the experiment, cells identified by the mutant probe (red fluorescence) were sorted. The sorted cells were then characterized to confirm the presence of the point mutation by PCR amplification of the gene segment containing the mutation followed by sequencing the PCR product. Cells expressing wild-type PU.1 were also be sorted and sequenced. These experiments provided independent evidence confirming the probes were detecting the correct sequences. Normal bone marrow cells alone were also analyzed in this manner. If the R235C mutation was detected at low frequency in normal bone marrow, even 1 cell in a million, the cells were sorted and sequenced for confirmation. This provides an example of the utility of the technique in detecting background mutations. It also provides an experimental means to detect false positives. Knowing the frequency of false positives is helpful in making an assessment of the suitability of any assay for the purpose of early cancer detection. It also sets a baseline frequency that is used in clinical applications of this technology to indicate the probability that a tumor is present. It is contemplated by the inventors that upon completion of the mixing experiments, experiments to detect additional phenotypes may be attempted. It is likely that these experiments may use numerous cells at the onset, because washing steps accomplished by centrifugation lead to the cell loss. The order of steps is likely to be critical. For example, it may be useful to allow immunoglobulins to bind to cell surface proteins prior to cross-linking with paraformaldehyde and fixation. Both immunophenotyping and FISH using the BAC probe to chromosome 2 may be attempted in cells hybridized with PNA-DNA probes.

### Example 4

### Determination of Rare Cell Detection Limit

### Rare Cell Detection Limit.

The limiting frequency with which rare cells can be detected can be experimentally determined and compared to theoretical expectations.

Either precancerous cells or cells from early stage cancers that contain a recurrent point mutation may be present at low frequency in biopsies. Therefore it may be useful to experimentally evaluate the detection limit of this technology and compare it to expectations based on statistical analysis. 8016 PU.1(del/R235C) cells are transfected with a plasmid containing the gene for GFP and selected using antibiotic resistance. A clone expressing GFP is expanded for use in this experiment, and evaluated to be certain its cancer-related characteristics have not changed compared to the original tumor-derived cell line. It is expected that all cells in the clone would be capable of expressing mRNA with the R235C mutation but may not express the message throughout the cell cycle. GFP protein is expected to be present at all times. To confirm this, GFP+8016 PU.1(del/R235C) cells are hybridized with mutant probe and visually inspected for green fluorescence (GFP) and red fluorescence (R235C mutation). It is contemplated that this experiment allows estimate the fraction of mutant cells that cannot be detected because they do not express R235C mRNA.

Normal bone marrow cells are mixed with GFP+8016 PU.1(del/R235C) cells in decade increments. The mixed cell populations, plus 100% mutant and 100% normal cell controls, are attached to glass slides using the cytocentrifuge protocol. A labeled mutant probe/unlabeled wild-type probe mixture is hybridized to the cells and analyzed using LSC. GFP+/PU.1−, GFP−/PU.1+ (if any), and double positive cells were scored. A fixed number of cells are analyzed from each sample and thresholds set as described above. The data is analyzed by one-tailed t-tests to determine if there is a statistically significant increase in the mean of samples containing various ratios of R235C mutant cells and normal cells compared to a control consisting of all normal cells. This experimentally determined detection limit may be compared to the theoretical expectation corresponding to the case where the same number of cells are analyzed, and no control cells test positive and no mutant cells escape detection.

It is contemplated that the experiment may be repeated with a probe mixture in which both probes are labeled with different fluorophores. This requires three-color detection and analysis, and allows mutant and normal cells to be assessed independently. There may be occasions where a cell is scored as mutant with one probe and normal with the other. The frequency with which this occurs provides a measure of the assay's ability to discriminate between mutant and normal cells. Cells that are labeled with GFP or by other means, and that are known to be either mutation-carrying or wild-type, may be useful in setting fluorescence threshold levels when the assay is applied to actual test samples. In this case, the test samples may be spiked with marked cells, hybridization performed, and data collected. Data from the marked cells would then be used to set thresholds for the entire sample.

### Example 5

### Detection of Multiple Cancer Phenotypes in Each Cell

Cancer cells have multiplegenotypic and resultant phenotypic changes. Determination of the molecular changes that have occurred may allow accurate diagnoses and the design of rational individualized treatments. It is contemplated that the point mutation assay can be combined with other types of molecular markers of cancer cells, DNA content, proteins detected using monoclonal antibodies, and a large chromosomal deletion detected using FISH may be combined with detection of the R235C point mutation in mRNA. Immunophenotyping is commonly used in precise diagnosis of human hematopoietic malignancies. Cell surface markers present on the AML-derived 8016 cells may be identified using monoclonal antibodies to mouse myeloid cell surface markers. The deletion of one copy of PU.1 from one mouse chromosome 2 homolog in 8016 PU.1(del/R235C) cells may be detected using a specific BAC probe and FISH (6). Then the conditions allowing these independent assays to be combined may be investigated using LSC. The goal is to demonstrate the combination of immunophenotyping, ploidy analysis based on DNA content, PU.1 deletion analysis, and detection of the R235C point mutation in the same cells. Because it requires thermal denaturation, it is contemplated that a second round of slide processing and analysis may be used to detect PU.1 deletions. Although cross-linking with paraformaldehyde might allow simultaneous antibody and FISH detections, a two step procedure may provide an opportunity to test LSC's ability to perform sequential assays and return to the same cells for analysis.

### Example 6

### Distinguishing Between Mutant and Normal Alleles in Heterozygous Cells

The inventors contemplate that a well characterized cell line having the activated ras mutation may be used to demonstrate the utility of the technique for the detection of recurrent point mutations in human cells. This cell line may be obtained and sequenced around the reported point mutation to confirm its presence. Cells that express both wild-type and mutant mRNAs may be used for LSC experiments. PNA-DNA probes to the wild-type and mutant sequences may be designed and labeled with different fluorophores. Hybridization conditions may be optimized as above prior to LSC. This experiment demonstrated an ability to detect, and discriminate between, wild-type RNA and mutant RNA when both are present within the same cell using PNA-DNA probes and the quantitative analytical methods described above. A human cell line expressing both wild-type and activated ras was hybridized on slides to a mixture of two labeled probes. Using LSC and the experimental and analytical methods developed in this project the assay's ability to discriminate between wild-type and mutant alleles when both are present within the same cell was demonstrated. All 6 slides were mutant cells. Slides 1-3 had the wild-type-green probe, with and without an unlabeled competitor probe. Slide 2 had unlabeled mutant probe at 1:1 and green was knocked down almost half. Slide 3 had unlabeled mutant probe at 1:10 and the green fluorescence was knocked down 1,600 fold. Slides 4-6 were the same but the mutant probe was labeled and the wild type competitor was not. There was little change in fluorescence when competing with WT probe.

### Sequences:

PU.1 mouse WT PNA-DNA
PNA portion GCC GTA GTT GCG CAG C- start
DNA portion GAT TAT TAT TAT TAC CAC TCA GAG GTT TTT TCC TCT GAG TGG
(NOTE THIS DNA SEQUENCE WILL BE COMMON TO ALL PROBES and will be henceforth denoted as—DNA)
   MELTING TEMP 60° C.
PU.1R235C mouse (most common)
   GCT GCG CAA CTA CGG C-DNA
   MELTING TEMP 60° C.
PU.1R235H mouse (second most common)
   TTG CCG TAG TTG TGC AGC-DNA
   MELTING TEMP 60° C.
PU.1 WT sense strand mouse—negative control will not bind to message
   GCT GCG CAA CTA CGG C-DNA
   MELTING TEMP 60° C.
KiRas human
   TGC CTA CGC CAC CAG C-DNA
   MELTING TEMP 60° C.
KiRas human
   TTG CCT ACG TCA CCA GCT-DNA
   MELTING TEMP 60° C.

When introducing elements or features of embodiments herein, the articles “a”, “an”, “the” and “said” are intended to mean that there are one or more of such elements or features. The terms “comprising”, “including”, and “having” are intended to be inclusive and mean that there may be additional elements or features other than those specifically noted. The phrase “consisting essentially of” is intended to limit the scope of a claim to the specified materials or steps and those that do not materially affect the basic and novel characteristic(s) of the claimed subject matter. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

The description of the disclosure is merely exemplary in nature and, thus, variations that do not depart from the gist of the disclosure are intended to be within the scope of the disclosure. Such variations are not to be regarded as a departure from the spirit and scope of the disclosure.

What is claimed is:

1. A method of identifying a point mutation in a cell, the method comprising:
(a) providing a cell obtained from a tissue sample;
(b) providing a first hybridization solution comprising probes complementary to a portion of a wild-type mRNA containing the site where the mutation is known to occur;
(c) providing a second hybridization solution comprising probes complementary to a portion of mutant mRNA containing the point mutation;
wherein the probes from at least one of steps (b) and (c) are labeled;
(d) incubating the cell with the first hybridization solution;
(e) incubating the cell with the second hybridization solution;
(f) detecting the amount of hybridized labeled probe in the cell relative to a wild-type control;
wherein a change in amount of hybridized labeled probe relative to a wild-type control identifies the presence of a point mutation in the gene.

2. The method of claim 1, further comprising providing a secondary marker for a cancerous cell, contacting the cells with the marker, and detecting the secondary marker, and wherein a positive identification of the secondary marker is made, the cell is identified as a cancer cell.

3. The method of claim 2, wherein the secondary marker is selected from the group consisting of a DNA dye, a labeled antibody to a protein of interest, and a DNA probe.

4. The method of claim 1, wherein the probes are PNA probes, LNA probes, modified RNA probes, modified DNA probes, chimeric PNA-DNA probes, chimeric LNA-DNA probes, or mixtures thereof.

5. The method of claim 1, wherein the probes from step (b) are labeled with a fluorescent tag, a biotinylated tag, or a hapten.

6. The method of claim 1, wherein the probes from step (b) and (c) are labeled with a different fluorescent tag, biotinylated tag, or hapten.

7. The method of claim 1, wherein the first hybridization solution and second hybridization solution are combined into one solution.

8. The method of claim 1, wherein the recurrent point mutation is a gain-of-function mutation.

9. The method of claim 1, wherein the recurrent point mutation is in a gene selected from the group consisting of H-Ras, N-Ras, and K-Ras.

10. The method of claim 1, wherein the detecting of the labeled probe is performed using laser scanning cytometry, FISH, or flow cytometry.

11. The method of claim 1, wherein steps (d) and (e) are performed sequentially.

12. The method of claim 1, wherein steps (d) and (e) are performed simultaneously.

13. The method of claim 11, wherein the step of detecting the amount of hybridized labeled probe is performed after each of steps (d) and (e).

14. A composition comprising labeled probes homologous to a wild-type mRNA from a gene known to be susceptible to a recurrent single point mutation and unlabeled probes homologous to mRNA from the gene containing a known single point mutation.

15. The composition of claim 14, wherein the probes are chimeric PNA-DNA probes.

16. The composition of claim 14, wherein the PNA portion of the probe is between 10 and 20 nucleotides.

17. The composition of claim 14, wherein the DNA portion of the labeled probe is a substrate for extension by an enzyme for labeling.

18. The composition of claim 15, wherein the labeled probe is labeled with a fluorophore.

19. A kit comprising:
(a) a first hybridization component comprising probes complementary to a portion of a wild-type mRNA containing the site where a mutation is known to occur; and
(b) a second hybridization component comprising probes complementary to a portion of mutant mRNA containing the point mutation.

20. The kit of claim 19, further comprising (c) a third component comprising a secondary marker for a cancerous cell.

21. The kit of claim 19, wherein the probes from (a) and the probes from (b) are labeled with two different fluorophores.

22. The kit of claim 19, wherein the probes from (a) are labeled with a fluorophore.

23. The kit of claim 19, wherein the first hybridization component and second hybridization component are combined into one container.

24. The kit of claim 19, wherein the recurrent point mutation is a gain-of-function mutation.

25. The kit of claim 20, wherein the third component contains a probe to detect deletion of a gene selected from the group consisting of p53, BRCA1, BRCA2, RB, and PTEN.

26. The kit of claim 20, wherein the third component contains a DNA dye selected from the group consisting of propidium iodide, 7-AAD, DAPI, Hoechst 33342 trihydrochloride trihydrate, SYBR Green I, YO-PRO-1, TOTO-3, and TO-PRO-3.

27. The kit of claim 20, wherein the third component contains an antibody to a protein selected from the group consisting of Her2 and Ca125.

28. A method of identifying a point mutation in a cell, the method comprising:
(a) providing a cell obtained from a tissue sample;
(b) providing a first set of labeled probes complementary to a portion of a wild-type mRNA containing the site where the mutation is known to occur;
(c) providing a second set of labeled probes complementary to a portion of mutant mRNA containing the point mutation;
wherein the probes from steps (b) and (c) are labeled with fluorophores of different colors;
(d) combining the first set and second set of labeled probes in a ratio of about 1:1 into one hybridization solution;
(e) incubating the cell with the hybridization solution;
(f) detecting the fluorescence intensity of the cell and compute the fluorescent intensity ratio of the cell;
wherein a change in the fluorescent intensity ratio relative to a wild-type control identifies the presence of a point mutation in the gene.


SEQUENCE LISTING (7 sequences)

SEQ ID NO: 1 - DNA, 16 bp, Artificial Sequence
  Synthetic Probe
gccgtagttgcgcagc

SEQ ID NO: 2 - DNA, 42 bp, Artificial Sequence
  Synthetic Probe
gattattattattaccactcagaggttttttcctctgagtgg

SEQ ID NO: 3 - DNA, 16 bp, Artificial Sequence
  Synthetic Probe
gctgcgcaactacggc

SEQ ID NO: 4 - DNA, 18 bp, Artificial Sequence
  Synthetic Probe
ttgccgtagttgtgcagc

SEQ ID NO: 5 - DNA, 16 bp, Artificial Sequence
  Synthetic Probe
gctgcgcaactacggc

SEQ ID NO: 6 - DNA, 16 bp, Artificial Sequence
  Synthetic Probe
tgcctacgccaccagc

SEQ ID NO: 7 - DNA, 18 bp, Artificial Sequence
  Synthetic Probe
ttgcctacgtcaccagct